(19) United States
(12) Patent Application Publication
(10) Pub. No.: US 2026/0325575 A1
(43) Pub. Date: Oct. 22, 2026

(54) THE USE OF BORATE DERIVATIVES IN THE TREATMENT OF NEURODEGENERATIVE DISEASES

(71) Applicant: YEDITEPE UNIVERSITESI, Istanbul (TR)

(72) Inventors: Fikrettin SAHIN, Istanbul (TR); Nermin Basak SENTURK, Istanbul (TR); Burcu KASAPOGLU USTA, Istanbul (TR); Sevda DEMIR, Istanbul (TR)

(73) Assignee: YEDITEPE UNIVERSITESI, Istanbul (TR)

(21) Appl. No.: 19/115,798

(22) PCT Filed: Sep. 28, 2022
(86) PCT No.: PCT/TR2022/051050

§ 371 Date: Mar. 27, 2025

(51) Int. Cl. A61K 33/22; A61K 9/08; A61P 25/28

(52) U.S. Cl. CPC A61K 33/22; A61K 9/08; A61P 25/28

(57) ABSTRACT

Provided is a use of borate derivatives in the treatment of amyotrophic lateral sclerosis (ALS), which is a neurodegenerative disease. More particularly, the development and administration of pharmaceutical formulations including at least one borate derivative is provided. The borate derivatives are one or a mixture of compounds sodium pentaborate pentahydrate (NaB; NaB₅O₈·5H₂O) or sodium perborate tetrahydrate (SPT; NaBO₃·4H₂O).

### CROSS REFERENCE TO THE RELATED APPLICATIONS

This application is the national phase entry of International Application No. PCT/TR2022/051050, filed on Sep. 28, 2022, the entire contents of which are incorporated herein by reference.

### TECHNICAL FIELD

The present invention relates to the use of borate derivatives in the treatment of amyotrophic lateral sclerosis (ALS), which is a neurodegenerative disease. More particularly, it relates to the development and administration of pharmaceutical formulations including at least one borate derivative disclosed in the said invention.

### BACKGROUND

Amyotrophic lateral sclerosis (ALS) is defined as a neurodegenerative disease characterized by progressive muscle paralysis reflecting degeneration of motor neurons in the primary motor cortex, brainstem and spinal cord. Critical connections between the Central nervous system and skeletal muscles are progressively lost, subsequently resulting in paralysis, respiratory failure and death [1,2].

At least 90% of ALS cases are of the sporadic type with no known familial inheritance pathway [3]. The other 10% of the cases can be associated with familial inheritance of a range of genetic mutations. The identification of mutations in the superoxide dismutase 1 (SOD1) gene in 1993 has triggered the first major molecular research wave in ALS [4]. SOD1 is a protein which is expressed in almost every cell type and catalyzes the detoxification of superoxide. More than 150 mutations in SOD1 are only associated with ALS. Transgenic mice overexpressing mutant SOD1 display an ALS-like phenotype and are considered as the cornerstone of ALS research.

Boron compounds have found many applications in our lives. In particular, they are used in cleaning and bleaching, dyes, toothpastes, textiles and textile dyes. Recent in vitro studies have proven that different boron derivatives can be effective in the treatment of obesity and in many treatment models such as wound healing thanks to their antibacterial/anti-inflammatory properties [5, 6, 7, 8]. Studies showing that boron derivatives inhibit the proliferation of cancer cells have demonstrated that the compounds thereof may be promising agents for cancer therapies [9, 10, 11, 12]. Studies on whether boron derivatives have therapeutic and/or preventive effects in neurodegenerative diseases are ongoing.

Currently, there are two drugs approved for the treatment of ALS. One of them is Riluzole, which has been on the market for 25 years and has been proven to extend patient's life span by approximately a few months [13], and the other is Edaravone, which was clinically approved in 2017 [14]. However, these drugs, which have become part of the routine treatment, unfortunately have not made a significant positive impact on the course of the disease and survival time.

Over the past years, more than 60 molecules have been investigated as a potential treatment for ALS, however, although there were promising drug candidates among these molecules, unfortunately, the vast majority of them failed to demonstrate sufficient clinical efficacy when phase studies were conducted [15]. Currently, the FDA-approved drugs used in the treatment of ALS have not been able to provide a significant improvement in the progression of the disease, as previously mentioned. Therefore, researches for developing novel drug candidates that aim to significantly improve the course of the disease are becoming increasingly important.

As is the case with most neurodegenerative diseases, it is estimated that some of the underlying causes of ALS are due to neuroinflammation [16]. The present invention is developed by focusing in our study on the possibility that boron compounds, which have been found to have antimicrobial and anti-inflammatory properties in the studies conducted in the healthcare field, may also be an effective agent in the treatment of ALS disease.

Accordingly, sodium pentaborate pentahydrate (NaB; NaB₅O₈·5H₂O) derivatives, whose antimicrobial, anti-inflammatory and wound healing properties have been proven in previous studies [7, 17], and sodium perborate tetrahydrate (SPT; NaBO₃·4H₂O) derivatives, which are still used as bleaching agents today [18], are used in the present invention.

Boron is a naturally occurring trace element with the Symbol B and atomic number five [19]. It is not present in nature in its elemental form, and usually forms organoboron complexes with oxygen and sodium. Boron is also an essential nutrient for many organisms. Especially, organoboron complexes are highly important for the biological activities of living organisms. In healthy tissues, boron is present in the form of boric acid or borate. Although its total amount in the body is about 3-20 mg, the amount varies according to the structure of the tissue [20-22].

Borates are odorless white crystals and dissolve immediately in water. The simplest borates are boron oxide (B₂O₃) and boric acid (H₂BO₃).

The Chemical name of sodium pentaborate pentahydrate (NaB; NaB₅O₈·5H₂O), which is a boron-based compound, is sodium; tetrakis(oxoboranyloxy)boranuide; pentahydrate (IUPAC).

The Chemical name of sodium perborate tetrahydrate (SPT; NaBO₃·4H₂O), which is a boron-based compound, is sodium; 3-oxydodioxaborirane; tetrahydrate (IUPAC).

The state-of-the-art patent documents (TR2019/08285, TR2018/03493) disclose the use of boron-based borate derivatives alone and/or in combination in the treatment of various diseases.

The state-of-the-art patent application numbered EP1790349A1 discloses the treatment of viral or bacterial infections with pentahydrated sodium pentaborate {NaB₅O₂·5H₂O}.

The state-of-the-art patent application numbered WO2014168592A1 discloses an antiviral gel containing sodium pentaborate pentahydrate and used in the treatment of infections related to the Herpes Simplex virus.

In the report on the estimated market size of drugs used in neurodegenerative diseases, 2019-2026, published in Fortune Business Insight, the neurodegenerative diseases drugs market is geographically categorized into five main regions. These regions can be listed as North America, Asia-Pacific, Latin America, Middle East & Africa and Europe, where Turkey is located. With the largest market size, the US, located in the North America, is expected to hold a market share of 62,786.2 million dollars in 2026.

### SUMMARY

The objective of the present invention relates to the use of boron-based borate derivatives in the treatment of amyotrophic lateral sclerosis (ALS).

More particularly, the present invention relates to the use of the compounds sodium perborate tetrahydrate and sodium pentaborate pentahydrate in the treatment of the ALS disease.

Another objective of the present invention relates to realizing and preparing a pharmaceutical formulation including at least one borate derivative mentioned above in the said invention.

A further objective of the present invention relates to developing pharmaceutical formulations including therapeutically effective amount of at least one pharmaceutically acceptable borate derivative and at least one pharmaceutically acceptable excipient, and to the use thereof as a drug in the treatment of amyotrophic lateral sclerosis (ALS).

### BRIEF DESCRIPTION OF THE DRAWINGS

“The Use of Borate Derivatives in the Treatment of Neurodegenerative Diseases” which is realized to achieve the objective of the present invention is illustrated in the accompanying figures, in which:

FIG. 1. is a graphical representation of the motor performances of the mouse groups before (t=0) and after administration of borate. Data represent the longest residence time of three test trials for each mouse (n=8 per group)±SEM (** P<0.01).

FIG. 2. is a representation of the gene expressions of the other groups compared to the transgenic control group in the form of a heat map, as a result of the microarray analysis performed on the spinal cord materials of the transgenic and wild type experimental groups (n=3 per group) that received NaB, SPT and water only. (Statistical differences between groups were compared using Volcano plot analysis (two-way ANOVA; P values<0.05) and fold change ±2 and more were considered statistically significant).

FIG. 3. is a representation of some of the pathways in which SPT and NaB derivatives are found to be effective as a result of the pathway analysis (PANTHER Gene Ontology (GO) Classification and Pathway System) and of the number of genes located in these pathways (Total number of genes: 2677, total number of hits: 443).

FIGS. 4A-4B. show a normal SHSY5Y cell (FIG. 4A) and a SHSY5U cell transformed into a cholinergic neurone (FIG. 4B).

FIG. 5. shows the gene expression levels in normal and differentiated SHST5Y cells.

FIGS. 6A-6B. show the non-toxic concentrations of amyloid beta and sodium pentaborate.

FIGS. 7A-7C. show the effect of sodium pentaborate treatment on amyloid beta plaques (FIG. 7A. Negative control, FIG. 7B. Amyloid beta only, FIG. 7C. Amyloid beta and sodium penta borate together).

FIG. 8. shows the effect of sodium pentaborate on amyloid beta plaques.

### DETAILED DESCRIPTION OF THE EMBODIMENTS

The subject matter of the invention relates to the use of boron-based borate derivatives as drugs for the treatment of neurodegenerative diseases, including at least one of Amyotrophic Lateral Sclerosis (ALS), Parkinson's disease, Huntington's disease, and Alzheimer's disease.

As used herein, the term “pharmaceutically acceptable” refers to the molecules or compounds that do not produce any adverse allergic effect or other undesirable reaction when administered to an animal or human.

As used herein, the term “excipient” refers to any pharmaceutically acceptable auxiliary substance such as a preservative, filler, lubricant, pH adjusting agent, disintegrant, surfactant, solvent, viscosity agent, emulsifier, dispersant, which can be used in pharmaceutical formulation studies.

As used herein, the term “treatment” refers to prevention or reduction or complete elimination of the occurrence or progression of the condition to which the term applies or of one or more symptoms of the said condition.

As used herein, the term “therapeutically effective amount” refers to the dosage of the drug that is administered to a patient in need of such treatment and that provides the specific pharmacological response.

The boron-based borate derivatives used in the treatment disclosed in the said invention are any one or a mixture of the compounds sodium pentaborate pentahydrate (NaB; NaB₅O₈·5H₂O) and sodium perborate tetrahydrate (SPT; NaBO₃·4H₂O).

The pharmaceutical formulations of the present invention can be prepared using Standard techniques or production methods known in the State of the art.

The pharmaceutical formulation of the present invention is formulated so as to be used in the treatment of amyotrophic lateral sclerosis (ALS).

According to the invention, the compounds sodium pentaborate pentahydrate (NaB) and sodium perborate tetrahydrate (SPT) can be administered orally, systemically, parenterally, nasally or enterally. In the preferred embodiment of the invention, the compound or compounds may be administered orally or enterally in a suitable formulation and dosage form. More specifically, the compounds are used as pharmaceutical formulations, preferably as Solutions, with at least one excipient during enteral administration. Herein, the preferred excipient is the solvent, which is basically water.

The strengths (therapeutically effective amounts to be received in one administration; dosage amount) of the compounds of the invention are adjusted so that they are effective in achieving the desired therapeutic response for a pharmaceutical form specific to the method of administration. Accordingly, dosages and excipients can be adjusted depending on the desired therapeutic effect, administration route, desired treatment duration and other criteria.

The specific dose level for any patient (human or mouse) can be adjusted on a treatment-specific basis depending on various criteria, including body weight, general health, gender, diet, duration and route of administration, intestinal absorption and excretion characteristics, combination with other drugs and severity.

In the preferred embodiment of the invention, the total daily dose of said compound or compounds for use in the treatment of ALS, administered in single or divided doses, may be in amounts of 0.001 to 200 mg/kg body weight per day for SPT and 0.001 to 1000 mg/kg body weight per day for NaB.

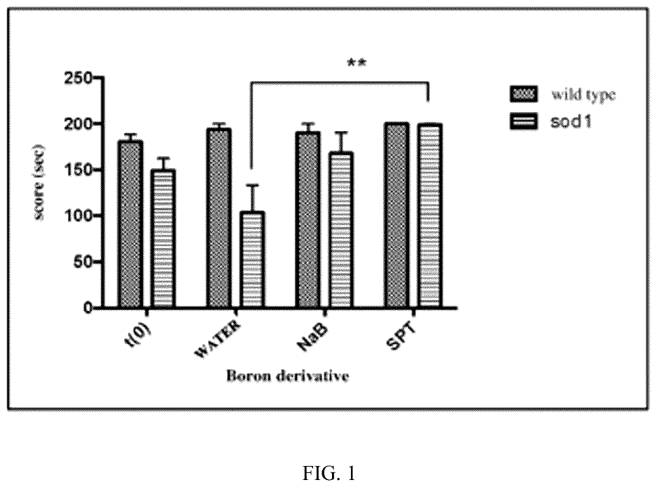

In the in vivo studies conducted, the SOD1 G93A transgenic mice model, which is one of the most preferred animal models for modeling ALS disease, was used. In the study (n=8); SPT and NaB were administered to the animals every other day by gavage. As a result of the comparison of the motor functions of these transgenic animals that received SPT and NaB throughout the experiment with the motor functions of the transgenic control (water) group, it was observed that SPT increased the performance of the rotarod test by 88.4% in the SOD1 G93A model compared to the control group (P<0.01). In the group that received NaB, there was a 62.45% increase in rotarod performance compared to the control group. In the group that received water, it was observed that the findings aggravated over time and the rotarod performance decreased. (shown in FIG. 1)

In the studies conducted in relation to the present invention, according to the neuroscoring system developed specifically for the SOD1 G93A transgenic mice model [24], it was observed that in the group that received SPT and NaB, animals that were still in stage 1 (NS1) at the end of the experiment showed milder classical symptoms of the stage such as hindlimb retraction, tremor and ataxia, and that these symptoms were considerably weaker than those in the control group. (Table 3)

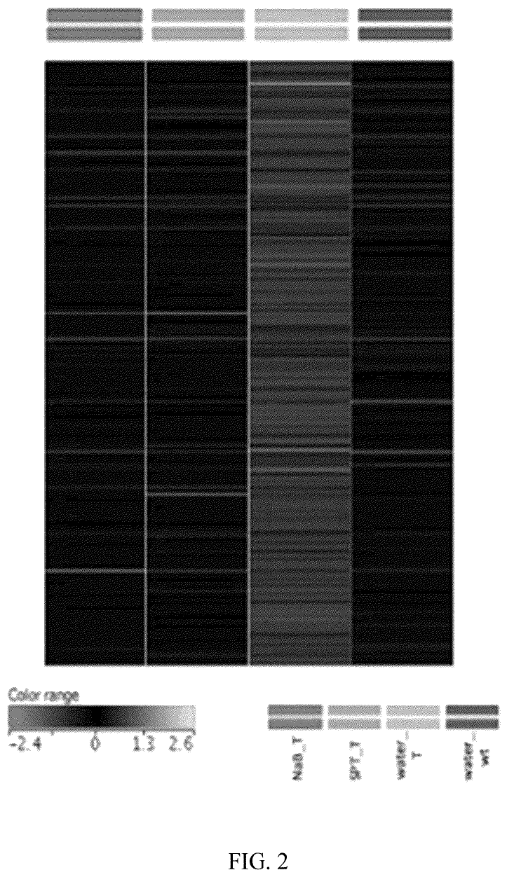

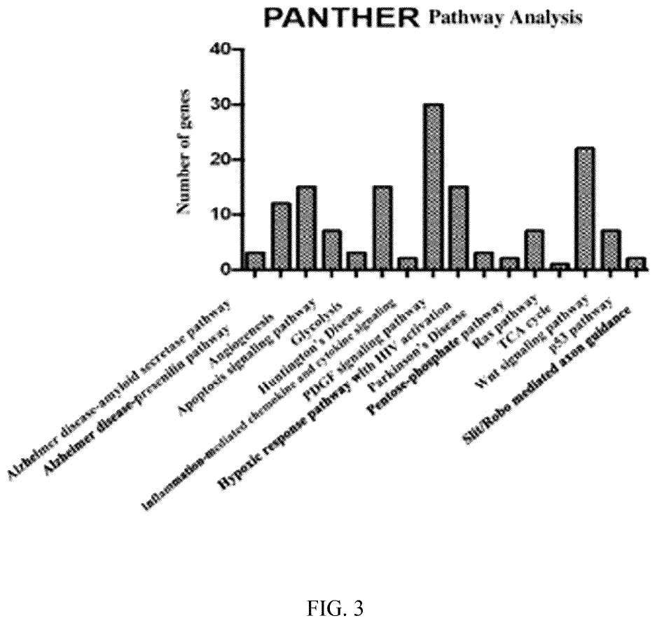

In addition to measurements of motor function, in order to investigate whether derivatives induce any gene-based changes in the Central nervous system, brain and spinal cord materials were collected from wild type and transgenic control (water), NaB and SPT group animals with n=3 and RNA was isolated from these materials and microarray analysis was performed. When the gene expression profiles of the groups were analyzed and represented as a heatmap as a result of the analysis, it was observed that in the transgenic control (water) groups, the expression of genes that are effective in many metabolic pathways in brain and spinal cord tissues changed compared to the wild type control group, but interestingly, in the transgenic animal groups that received SPT and NaB, the derivatives were effective in these pathways and reversed the gene expression at a statistically significant rate, approximating it to the gene expression level in wild type animals (shown in FIG. 2). Furthermore, as a result of the pathway analysis (PANTHER Gene Ontology (GO) Classification and Pathway System), it was observed that the derivatives were effective in the expression of genes associated with many metabolic pathways and caused positive changes in the expression of genes involved in pathways related to neurodegenerative diseases such as Parkinson's disease, Huntington's disease and Alzheimer's disease, especially in transgenic control group animals (shown in FIG. 3).

The positive findings obtained as a result of this application, which lasted for a very short period of approximately 21 days, indicate that borate applications for longer terms may cause much more effective changes and constitute a prelude for future clinical studies.

The following examples are to better illustrate the subject matter of the invention which is not limited to these examples.

### EXAMPLES

### Example 1 Information about the Experimental Animals Used

In the experimental studies carried out to achieve the present invention, the mouse models to which the compounds were administered are as follows:

TABLE 1
Experimental Animals

| Species | Breed | Sex | Age | Number |
|---|---|---|---|---|
| Mouse | C57BL/6 (wild type) | Female | 75-80 days old | 24 |
| Mouse | C57BL/6 (SOD1 | Female | 75-80 days old | 24 |
| | G93A, transgenic) | | | |

### Example 2 Experimental Groups

In the experimental studies carried out to achieve the present invention, the experimental animals were divided into groups and modeled as follows according 5 to the compound or control (water) group to be administered:

TABLE 2
Experimental Groups

| Groups | Animal | Substance to be |
|---|---|---|
| (n = 8) | species | administered |
| Group I | SOD1 G93A, | Control (water) |
| | transgenic | |
| Group II | SOD1 G93A, | Sodium perborate |
| | transgenic | tetrahydrate (SPT) |
| Group III | SOD1 G93A, | Sodium pentaborate |
| | transgenic | pentahydrate (NaB) |
| Group IV | Wild type | Control (water) |
| Group V | Wild type | Sodium perborate |
| | | tetrahydrate (SPT) |
| Group VI | Wild type | Sodium pentaborate |
| | | pentahydrate (NaB) |

### Example 3 Substance Administration

SPT or NaB derivatives at 200 mg/kg per mouse were vortexed at high speed and dissolved in the daily drinking water of the mice. Borate Solutions prepared thus in drinking water were administered to mice by gavage every other day for 3 weeks. The control group received only the same amount of drinking water by gavage.

### Example 4 Motor Function Tests

In order to understand whether the borate derivative administered caused a difference in the motor coordination of the animals, especially the transgenic mice, the animals were subjected to some motor function tests before and after administration.

### a) Rotarod Test:

The mice were acclimatized with the rotarod test apparatus for one day before their motor function was tested and their performance was recorded at the end of the second day. Their motor performances were measured three times for a maximum of 180 seconds at a constant speed of 14 rpm [25], and their longest residence time on the apparatus was recorded.

### b) Neuroscoring:

In order to observe during the experiment the stages when early and late symptoms of the disease occur, wild-type and transgenic animals were subjected to neuroscoring as described in the study by Hatzipetros et al [24]. In this way, it was aimed to observe whether the administration to the animals caused any difference in the onset, course and severity of ALS symptoms. For this, the table of the disease course is graded with a scoring System as follows:
1. Neuro Score 0 (NS 0, Pre-symptomatic): When suspended by the tail, the mouse can fully extend its hindlimbs away from the lateral midline to the side in a normal manner and stay in this position for at least two seconds or longer. When the mouse is allowed to walk, normal gait is observed.
2. NS 1 (First symptoms): When the mouse is suspended by the tail, its hindlimbs cannot fully extend away from the lateral midline, they partially collapse backwards or the hindlimbs are observed to tremble (tremor). Towards the end of the phase, gait disturbances and slow gait (ataxia) begin. The first symptoms usually appear as of day 90.
3. NS 2 (Partial paralysis, paresis): When the mouse is suspended by the tail, its hindlimbs are partially or completely collapsed, or cannot extend much, there is still joint movement. When the mouse is allowed to walk, it can use its hindlimbs for forward motion, however it is observed that its toes curl backwards at least twice during a 90 cm walk or it can move by dragging its feet. When the mouse is placed on its left or right side, it is able to right itself within 10 seconds.
4. NS 3 (Paralysis): When the mouse is suspended by the tail, it is observed that there is rigid paralysis in its hindlimbs or minimal joint movement. When the mouse is allowed to walk, it is observed that there is forward motion however it cannot use its hindlimbs. When the mouse is placed on its left or right side, it is observed that it is able to right itself within 10 seconds.
5. NS 4 (Humane end-point): When the mouse is suspended by the tail, it is observed that there is rigid paralysis in its hindlimbs. When the mouse is allowed to walk, no motion is observed, when the mouse is placed on its left or right side it is observed that it is not able to right itself within 10 seconds, i.e., absence of righting reflex.

According to this scoring system, if we divide the symptoms observed in animals into phases according to the order of occurrence, 50-80 days after birth can be considered pre-symptomatic. Since as of day 90 the first symptoms start to appear, we can consider this date as the beginning of the early symptomatic phase and also the beginning of neuroscore 1. Although it can vary, this period can last 20-38 days and also covers the neuroscore 2 phase. As of day 120 on average, the process is called late symptomatic. Although it may last a little longer in females, this period lasts 5-10 days on average and neuroscore 3 and 4 phases are also observed during this late symptomatic period [24]

TABLE 3
Detailed comparison of phenotypic characteristics of NS1 phase in experimental
and control groups before and after the administration of borate derivatives

| | day 80 (t = 0) | | | day 90 (onset of the disease-NS1) | | | day 110 (experiment endpoint) | | |
|---|---|---|---|---|---|---|---|---|---|
| | s1 | s2 | s3 | s1 | s2 | s3 | s1 | s2 | s3 |
| w.t. | — | — | — | — | — | — | — | — | — |
| tg- | — | — | very mild | moderate | moderate | moderate | severe | severe | severe |
| water | | | | | | | | | |
| tg- | — | — | very mild | very mild | very mild | very mild | mild | mild | mild |
| SPT | | | | | | | | | |
| tg- | | | very mild | mild | mild | mild | moderate | moderate | moderate |
| NaB | | | | | | | | | |

(w.t.: wild type, tg.: transgenic, s: symptom, sİ: hindlimb retraction, s2: tremor, s3: ataxia)

### Statistical Methods and Data Used:

### Method: ANOVA and T-Test

α: 0.05 (Type 1 Error)
σ: 0.11 (Standard deviation between groups to be compared)
δ: 0.8 (Difference in population)
m: 1 (ratio of the control to other groups)
n: 8

### Evaluation of the Efficacy of Sodium Pentaborate Against Alzheimer's Disease

The inventors are investigating the effectiveness of sodium pentaborate (NaB) as a boron derivative for Alzheimer's Disease, another neurodegenerative disease. Their test procedures are as follows:

### Obtaining Cell Culture

SHSY5Y cells stocked in cryo tubes at −80° C. or liquid nitrogen were kept in a water bath at 37° C. until completely solved. The solved cells were transferred to 15 ml Falcon tubes containing 5 ml cell medium and centrifuged at 300×g for 5 minutes. After centrifugation, the medium on the cells was removed and the cells were solved with 1 ml of fresh medium using a pipette. Then, the cells were transferred to cell flask containing 12 ml DMEM/F12 medium and incubated at 37° C. for 24±2 hours in an incubator containing 5% CO₂.

The next day, the medium on the adherent cells was changed and incubation was continued until the cells formed 85%-90% monolayer by weight. The medium of the monolayered cells was removed and the cells were washed with 5 ml PBS. Then, 3 ml of 0.25% trypsin/EDTA was added to the flask and incubated until the cells were completely removed. To the cells removed from the surface of the flask, 5 ml of medium was added and all cells were placed in a Falcon tube and centrifuged at 300×g for 5 minutes. Finally, half of the obtained cells were seeded in a new flask and incubated at 37° C. in an incubator containing 5% CO₂.

Differentiation of SHSY5Y Cells into Neuron Cells

SH-SY5Y cells were seeded in 6-well plates with 250×10⁵ cells in each well and incubated with medium containing DMEM/F12, 10% FBS, 1% PSA at 37° C. in 5% CO₂ for 24 hours. Then, the medium on the cells was removed and each well was washed with 1 ml PBS. Then, 2 ml of differentiation medium (1% FBS, 10 μM/ml RA) was added to each cell well and cells were incubated for 72 hours.

After the incubation period was completed, the medium on the cells was removed and the cells were washed again with 1 ml PBS. Afterwards, 2 ml of differentiation medium (1% FBS, 10 μM/ml RA, 10 μg/ml BDNF) was added to the cells and incubated for 72 hours. The same procedure was repeated on the seventh day. At the end of the tenth day, neuronal cells were ready.

### Characterisation of Neuron Cells

For characterisation purposes, the expression of acetylcholinesterase (AChE), choline acetyltransferase (ChAT), choline transporter 1 (CHT1) and vesicular acetylcholine transporter (VAChT) genes were examined. For this purpose, RNA was isolated from differentiated cells. Firstly, cells were detached from the surface with trypsin and washed three times with PBS. The cell pellet obtained was incubated for 5 minutes by adding 400 μl Trizol. Then, 80 μl of cold chloroform was added and centrifuged at 12,000×g for 15 minutes. The clear supernatant formed after centrifugation was transferred to a new tube, 400 μl isopropanol was added and incubated for 10 min. The mixture was centrifuged at 12,000×g for 10 minutes. The RNA pellet obtained was washed with 75% ethanol to remove excess salts. After drying, 50 μl of nuclease-free water was added to the pellet and RNA amount was measured by nanodrop device.

After RNA isolation was completed, cDNA synthesis from RNA was performed according to the kit protocol (iScript cDNA synthesis kit, Biorad). The obtained cDNAs were made up to 100 μl and subjected to RT-PCR. The reaction mixture was prepared by adding 2.5 μl SYBR green, 1 μl primer (F+R), 1 μl cDNA and 5.5 μl nuclease-free water to a 96-well PCR plate with a total volume of 10 μl. The prepared plate was placed in the ABI StepOnePlus device and RT-PCR was performed.

### Cytotoxicity Analysis

The maximum non-toxic dose of NaB and Amyloid Beta (Aβ) in neuronal cells was measured by MTS (3-(4,5-Dimethylthiazol-2-yl)-5-(3-carboxy methoxyphenyl) method depending on mitochondrial dehydrogenase enzyme activity. Within the scope of the experiment, cells were treated with different concentrations of Amyloid Beta (6, 4, 2, 1 and 0.5 μg/ml) and NaB (100, 50, 20, 10 and 5 μg/ml).

After the incubation period was completed (24 hours), the medium on the cells was removed and replaced with 10% MTS solution prepared in the cell medium. Cells were incubated at 37° C. in 5% CO₂ medium for two hours. After incubation, absorbance measurements were made using an ELISA plate reader at 490 nm. From the absorbance values obtained, cell viability was calculated according to the formula ((Mean Cell Abs./Mid. Negative Con. Abs.)*100).

### Obtaining Alzheimer's Model and Evaluation of Active Substances

On the differentiated cells, 2 ml of Amyloid Beta, the maximum non-toxic dose of which was determined and prepared in neuronal medium, was added and incubated for 24 hours at 37° C., 5% CO₂. After completion of the incubation period, Amyloid Beta on the cells was removed and the cells were washed three times with 1 ml PBS. Then, the maximum non-toxic dose of sodium penta borate was prepared in 2 ml neural media, added to the cells and incubated for 24 hours at 37° C., 5% CO₂.

### Immunocytochemistry Analysis

The old media on the incubated cells were removed and the cells were washed three times with 1 ml PBS. Cells were labelled according to the experimental groups: NK (Negative Control), OAβ (Amyloid Beta Only), ONaB (Sodium Penta Borate Only), Aβ+NaB (Amyloid Beta and Sodium Penta Borate together).

Firstly, cells were fixed with 1 ml of 2% paraformaldehyde for 30 min at room temperature and then washed three times with PBS. To increase the membrane permeability of the cells, 750 μl of 0.1% Triton X-100 was added to the cells and incubated at room temperature for 10 min. Then, 5% BSA was added to the cells and incubated at room temperature for 30 minutes.

After the blocking process was completed, the liquid on the cells was removed and Amyloid Beta antibody (Cell Signaling, #12843) prepared at a ratio of 1:200 in 5% BSA was added and the cells were incubated overnight at 4° C. After the incubation period was completed, the cells were washed three times with PBS. Then, secondary antibody (AlexaFluor 488, Thermo) prepared at a ratio of 1:1000 was added and the cells were incubated for 1 hour at room temperature and in the dark.

After incubation, the cells were washed three times with PBS. For nuclei staining, DAPI (Abcam, #ab285390) prepared at a ratio of 1:1000 was added to the cells and incubated for 5 min at room temperature in the dark. Finally, the cells were washed three times with PBS and measurements were taken with a fluorescence microscope (Zeiss).

### Data Evaluation

The data obtained from the cytotoxicity analysis were evaluated with MS Excel. The data obtained from the immunocytochemistry study were analysed using Image J (National Institutes of Health) and MS Excel.

### Results Obtained

Differentiation of SHSY5Y Cells into Neuron Cells

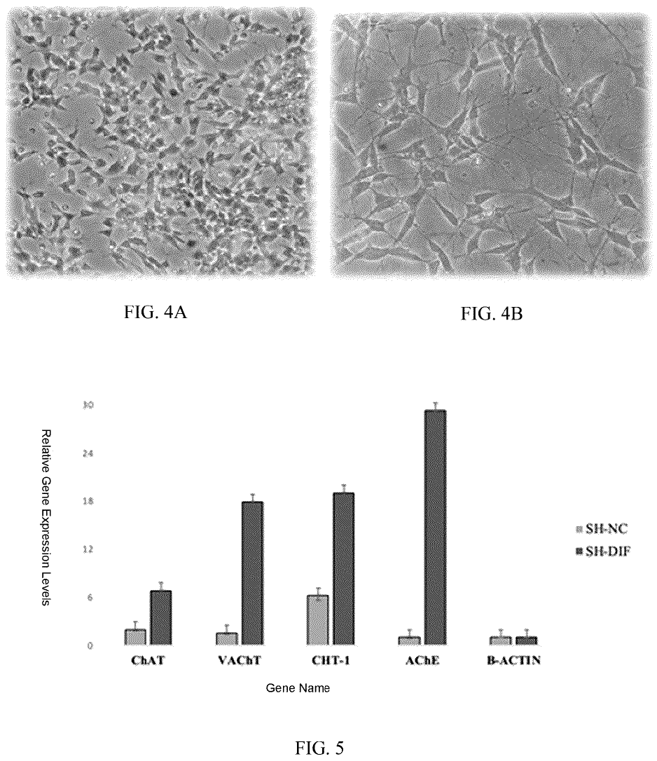

SH-SY5Y cells have a stable karyotype consisting of 47 chromosomes and can be differentiated from a neuroblast-like state into mature human neurons through a variety of different mechanisms, including retinoic acid, phorbol esters and brain-derived specific neurotrophic factors such as BDNF. Previous studies have shown that SH-SY5Y cells can differentiate into adrenergic, cholinergic and dopaminergic neurones depending on the method applied. Therefore, SH-SY5Y cells were preferred in this study in order to obtain cholinergic neurones. The neurones obtained after the 10-day protocol are shown in FIGS. 4A-4B.

### Characterisation of Neuron Cells

Cholinergic neurons are found in the central (brain and spinal cord) and peripheral (motor neurons and autonomic nervous system) nervous system and use a neurotransmitter called acetylcholine in nerve transmission. Acetylcholine is active in regulating muscle contractions and other neural transmissions. Cholinergic neurons play an important role in cognitive processes such as learning, memory, attention and focus, and acetylcholine is critically involved in the regulation of these processes. Therefore, for the characterisation of cholinergic neurons, ChAT and VAChT genes encoding enzymes and proteins critical for acetylcholine synthesis and transport, CHT1 gene involved in the reuptake of choline into the neuron and AChE gene responsible for the degradation of acetylcholine in the synaptic gap were identified and characterisation studies were performed. When gene expression levels were compared between cells without differentiation protocol and cells with differentiation protocol, it was found that there was a 5-fold increase for ChAT gene, 16-fold increase for VAChT gene, 13-fold increase for CHT1 gene and 28-fold increase for AChE gene in terms of gene expressions in differentiated cells (FIG. 5). This indicates that the cells gained cholinergic neuron character after the differentiation protocol applied to the cells.

### Cytotoxicity Analysis

Cytotoxicity assay is used to determine toxic and non-toxic concentrations of compounds. In this study, the non-toxic concentrations of Amyloid beta and sodium pentaborate were determined by MTS analysis.

This colorimetric analysis is based on the reduction of the MTS tetrazolium compound to the coloured and water-soluble formazan product by NAD (P) H-dependent dehydrogenase enzymes in metabolically active living cells.

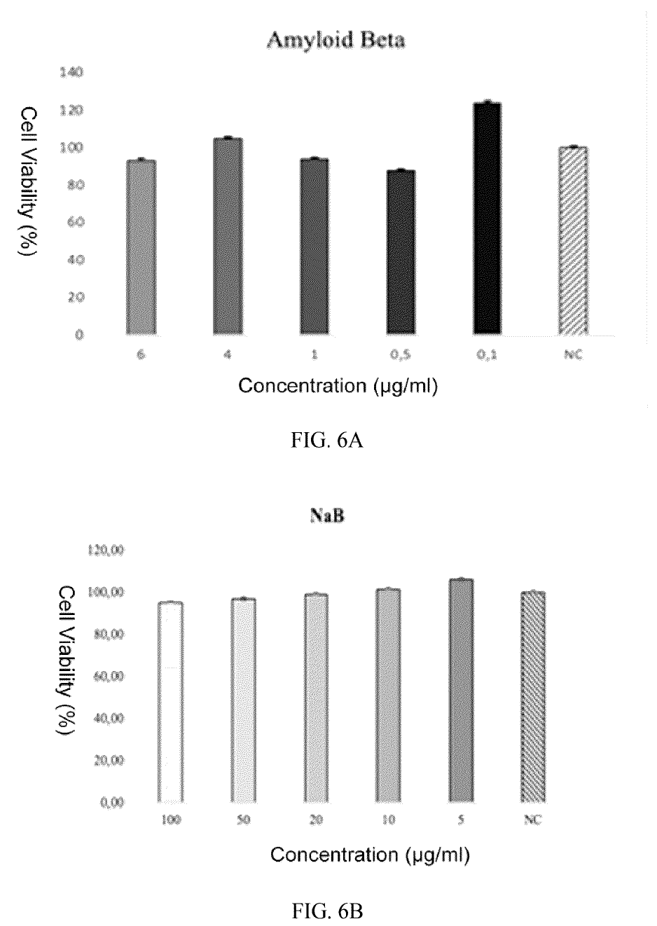

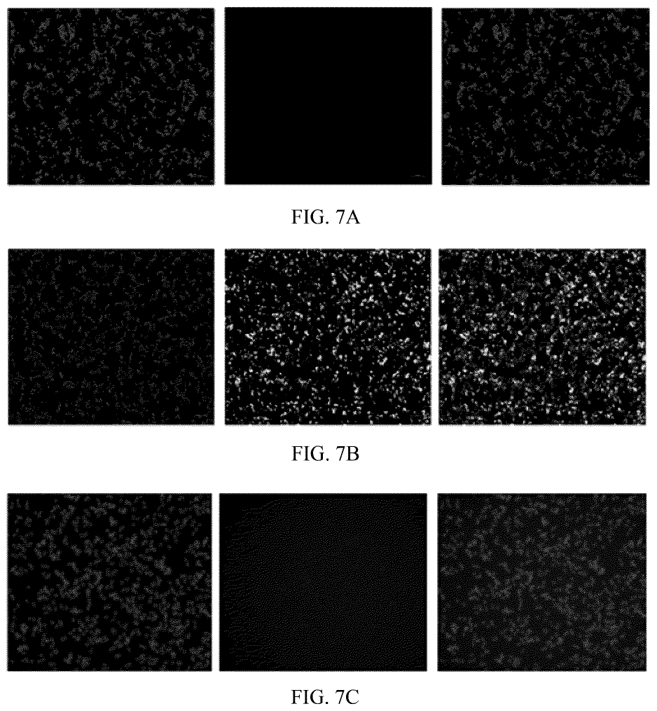

According to the results of the analyses, the non-toxic concentration of Amyloid Beta was determined as 4 μg/ml and the non-toxic dose of Sodium Penta Borate was determined as 5 μg/ml (shown in FIGS. 6A-6B).

### Evaluation of Alzheimer's Model and Trial of Active Substances

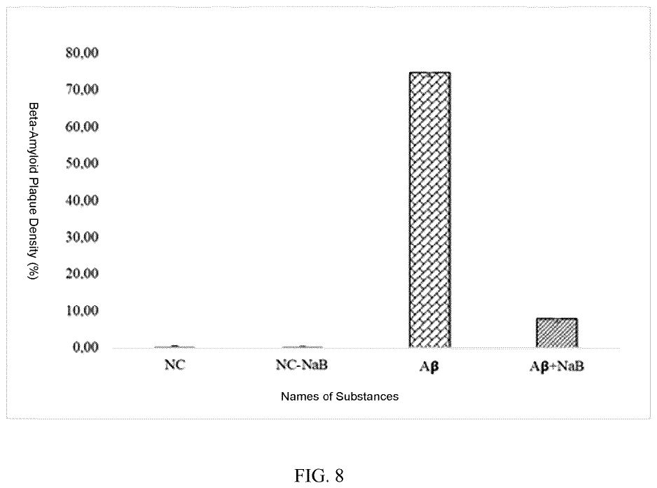

In order to create an Alzheimer's model, Amyloid beta was applied to SHSY5Y cells differentiated into cholinergic neurons for 24 hours and Amyloid beta plaques were formed in the cells. The formed plaques were determined by fluorescent staining technique. While 0% plaques were detected in NK and ONaB groups, 75% of the cells in OAB group formed Amyloid beta plaques. When only 5 μg/ml NaB was administered to the cells in which Amyloid beta plaques were formed for 24 hours, 92.42% of the plaques formed in the Aβ+NaB group dissolved compared to the OAβ group. The results obtained show that sodium penta borate has the potential to treat Alzheimer's disease via Aß plaques. The results are shown in FIG. 8 with a column graph.

### REFERENCES

[1]. Munsat, T. L., Andres, P. L., Finison, L., Conlon, T., & Thibodeau, L. (1988). The natural history of motoneuron loss in amyotrophic lateral sclerosis. Neurology, 38(3), 409-409.
[2]. Swash, M., & Schwartz, M. S. (1995). Motor neuron disease: the clinical syndrome. In Motor Neuron Disease (pp. 1-17). Springer, London
[3]. Taylor, J. P., Brown, R. H., & Cleveland, D. W. (2016). Decoding ALS: from genes to mechanism. Nature, 539(7628), 197-206.
[4]. Rosen, D. R., Siddique, T., Patterson, D., Figlewicz, D. A., Sapp, P., Hentati, A., . . . & Brown, R. H. (1993). Mutations in Cu/Zn superoxide dismutase gene are associated with familial amyotrophic lateral sclerosis. Nature, 362(6415), 59-62.
[5]. Argin, S., Gülerim, M., & Şahin, F. (2019). Development of antimicrobial gelatin films with boron derivatives. Turkish Journal of Biology, 43(1), 47-57.
[6]. Benderdour, M., Van Bui, T., Hess, K., Dicko, A., Belleville, F., & Dousset, B. (2000). Effects of boron derivatives on extracellular matrix formation. Journal of trace elements in medicine and biology, 14(3), 168-173.
[7]. Demirci, S., Kaya, M. S., Doğan, A., Kalay, Ş., Altin, N. Ö., Yarat, A., & Şahin, F. (2015). Antibacterial and cytotoxic properties of boron-containing dental composite. Turkish Journal of Biology, 39(3), 417-426.
[8]. Doğan, A., Demirci, S., Apdik, H., Bayrak, O. F., Gulluoglu, S., Tüysüz, E. C., & Şahin, F. (2017). A new hope for obesity management: Boron inhibits adipogenesis in progenitor cells through the Wnt/Ş-catenin pathway. Metabolism, 69, 130-142.
[9]. Das, B. C., Thapa, P., Karki, R., Schinke, C., Das, S., Kambhampati, S., & Evans, T. (2013). Boron Chemicals in diagnosis and therapeutics. Future medicinal chemistry, 5(6), 653-676.
[10]. Pike, A. C., Brzozowski, A. M., Hubbard, R. E., Bonn, T., Thorsell, A. G., Engström, O., & Carkprist, M. (1999). Structure of the ligand-binding domain of oestrogen receptor beta in the presence of a partial agonist and a full antagonist. The EMBO journal, 18(17), 4608-4618.
[11]. I Scorei, R., & Popa, R. (2010). Boron-containing compounds as preventive and chemotherapeutic agents for cancer. Anti-Cancer Agents in Medicinal Chemistry (Formerly Current Medicinal Chemistry-Anti-Cancer Agents), 10(4), 346-351.
[12]. Wang, Y., Zhao, Y., & Chen, X. (2008). Experimental study on the estrogen-like effect of boric Acid. Biological trace element research, 121(2), 160-170.
[13]. Favero, F. M., Voos, M. C., Castro, I. D., Caromano, F. A., & Oliveira, A. S. B. (2017). Epidemiological and clinical factors impact on the benefit of riluzole in the survival rates of patients with ALS. Arquivos de neuro-psiquiatria, 75(8), 515-522.
[14]. Cruz, M. P. (2018). Edaravone (Radicava): a novel neuroprotective agent for the treatment of amyotrophic lateral sclerosis. Pharmacy and Therapeutics, 43(1), 25.
[15]. Petrov, D., Mansfield, C., Moussy, A., & Hermine, O. (2017). ALS clinical trials review: 20 years of failure. Are we any closer to registering a new treatment?. Frontiers in aging neuroscience, 9, 68
[16]. Philips, T., & Robberecht, W. (2011). Neuroinflammation in amyotrophic lateral sclerosis: role of glial activation in motor neuron disease. The Lancet Neurology, 10(3), 253-263.
[17]. Doğan, A., Demirci, S., Çağlayan, A. B., Kiliç, E., Günal, M. Y., Uslu, Ü., & Şahin, F. (2014). Sodium pentaborate pentahydrate and pluronic containing hydrogel increases cutaneous wound healing in vitro and in vivo. Biological trace element research, 162(1), 72-79.
[18]. Weiger, R., Kuhn, A., & Löst, C. (1993). Effect of various types of sodium perborate on the pH of bleaching agents. Journal of endodontics, 19(5), 239-241
[19]. World Health Organization, Agency IAE, Nations & F and AO of the U. Trace elements in human nutrition and health World Health Organization. World Health Organization. Geneva, Switzerland; 1996.
[20]. Nielsen FH. Boron in human and animal nutrition. Plant Soil. 1997; 193(1): 199-208.
[21]. Penland J. The importance of boron nutrition for brain and psychological function. Biol Trace ElemRes. 1998; 66(1-3): 299-317.
[22]. Sutherland B, Strong P, King J. Determining human dietary requirements for boron. Biol Trace ElemRes. 1998; 66(1-3): 193-204.
[23]. Fortune Business Insights (2019). Neurodegenerative Diseases Drugs Market Size, Share and Industry Analysis By Drug Class (Immunomodulator, Interferons, Decarboxylase Inhibitors, Dopamine Agonists, Others), By Disease Indication (Multiple Sclerosis, Parkinson's Disease, Alzheimer's Disease, Spinal Muscular Atrophy (SMA), Others), By Route of Administration (Oral, Injection, Transdermal, Others), By End User and Regional Forecast 2019-2026. (No. FBI100661) www.fortunebusinessinsights.com/industry-reports/neurodegenerative-diseases-drugs-market-100661
[24]. Hatzipetros, Theo, et al. “A Quick Phenotypic Neurological Scoring System for Evaluating Disease Progression in the SOD1-G93A Mouse Model of ALS.” Journal of Visualized Experiments, no. 104, 2015.
[25]. Olivân, S., CAlvO, A. C., RAndO, A., MunOz, M. J., Zaragoza, P., & Osta, R. (2014). Comparative study of behavioural tests in the SOD1G93A mouse model of amyotrophic lateral sclerosis. Experimental animals, 14-0077.

What is claimed is:

1. A method for treating a neurodegenerative disease, comprising using one or a mixture of compounds sodium pentaborate pentahydrate (NaB; NaB₅O₈·5H₂O) or sodium perborate tetrahydrate (SPT; NaBO₃·4H₂O) as a drug.

2. The method according to claim 1, wherein the neurodegenerative disease is amyotrophic lateral sclerosis (ALS).

3. (canceled)

4. The method according to claim 1, wherein the drug comprises at least one excipient.

5. The method according to claim 4, wherein the at least one excipient is selected from the group consisting of a pharmaceutically acceptable preservative, a filler, a lubricant, a pH adjusting agent, a disintegrant, a surfactant, a solvent, a viscosity agent, an emulsifier, and a dispersant.

6. The method A use according to claim 4, wherein the at least one excipient is a solvent.

7. The method according to claim 1, wherein the drug is administered by one of oral, systemic, parenteral, nasal, and enteral routes.

8. The method according to claim 1, wherein an administered daily dose of the drug is 0.001 to 200 mg/kg body weight per day for the SPT and 0.001 to 1000 mg/kg body weight per day for the NaB.

9. The method according to claim 2, wherein the drug comprises at least one excipient.

10. The method according to claim 5, wherein the at least one excipient is the solvent.

11. The method according to claim 2, wherein the drug is administered by one of oral, systemic, parenteral, nasal, and enteral routes.

12. The method according to claim 4, wherein the drug is administered by one of oral, systemic, parenteral, nasal, and enteral routes.

13. The method according to claim 5, wherein the drug is administered by one of oral, systemic, parenteral, nasal, and enteral routes.

14. The method according to claim 6, wherein the drug is administered by one of oral, systemic, parenteral, nasal, and enteral routes.

15. The method according to claim 2, wherein an administered daily dose of the drug is 0.001 to 200 mg/kg body weight per day for the SPT and 0.001 to 1000 mg/kg body weight per day for the NaB.

16. The method according to claim 4, wherein an administered daily dose of the drug is 0.001 to 200 mg/kg body weight per day for the SPT and 0.001 to 1000 mg/kg body weight per day for the NaB.

17. The method according to claim 5, wherein an administered daily dose of the drug is 0.001 to 200 mg/kg body weight per day for the SPT and 0.001 to 1000 mg/kg body weight per day for the NaB.

18. The method according to claim 6, wherein an administered daily dose of the drug is 0.001 to 200 mg/kg body weight per day for the SPT and 0.001 to 1000 mg/kg body weight per day for the NaB.

19. The method according to claim 7, wherein an administered daily dose of the drug is 0.001 to 200 mg/kg body weight per day for the SPT and 0.001 to 1000 mg/kg body weight per day for the NaB.

20. The method according to claim 9, wherein the at least one excipient is selected from the group consisting of a pharmaceutically acceptable preservative, a filler, a lubricant, a pH adjusting agent, a disintegrant, a surfactant, a solvent, a viscosity agent, an emulsifier, and a dispersant.

21. The method according to claim 9, wherein the at least one excipient is a solvent.
